(19) United States
(12) Patent Application Publication
(10) Pub. No.: US 2021/0290170 A1
(43) Pub. Date: Sep. 23, 2021

(54) Detection of Motion Artifacts in Signals Output by Detectors of a Wearable Optical Measurement System

(71) Applicant: HI LLC, Los Angeles, CA (US)

(72) Inventors: Katherine Perdue, Los Angeles, CA (US); Ryan Field, Culver City, CA (US); Husam Katnani, Braintree, MA (US); Jennifer Rines, Carlsbad, CA (US); Alejandro Ojeda, Culver City, CA (US)

(21) Appl. No.: 17/202,631

(22) Filed: Mar. 16, 2021

Related U.S. Application Data
(60) Provisional application No. 62/992,512, filed on Mar. 20, 2020.
(60) Provisional application No. 63/051,099, filed on Jul. 13, 2020.

(51) Int. Cl. A61B 5/00

(52) U.S. Cl. CPC A61B 5/7214; A61B 5/0082; A61B 2576/026; A61B 2562/0238; A61B 2562/046; A61B 5/6803

(57) ABSTRACT

An illustrative system includes a wearable assembly configured to be worn by a user and comprising a first detector configured to detect a first set of photon arrival times and output a first signal representative of the first set of photon arrival times, and a second detector configured to detect a second set of photon arrival times and output a second signal representative of the second set of photon arrival times. The system further includes a processing unit configured to identify a first intensity change in the first signal, identify a second intensity change in the second signal, determine that the first and second intensity changes are time correlated, and determine, based on the determining that the first and second intensity changes are time correlated, that the first and second intensity changes are representative of a motion artifact caused by movement of the user.

### RELATED APPLICATIONS

The present application claims priority under 35 U.S.C. § 119(e) to U.S. Provisional Patent Application No. 62/992,512, filed on Mar. 20, 2020, and to U.S. Provisional Patent Application No. 63/051,099, filed on Jul. 13, 2020. These applications are incorporated herein by reference in their respective entireties.

### BACKGROUND INFORMATION

Detecting neural activity in the brain (or any other turbid medium) is useful for medical diagnostics, imaging, neuroengineering, brain-computer interfacing, and a variety of other diagnostic and consumer-related applications. For example, it may be desirable to detect neural activity in the brain of a user to determine if a particular region of the brain has been impacted by reduced blood irrigation, a hemorrhage, or any other type of damage. As another example, it may be desirable to detect neural activity in the brain of a user and computationally decode the detected neural activity into commands that can be used to control various types of consumer electronics (e.g., by controlling a cursor on a computer screen, changing channels on a television, turning lights on, etc.).

Neural activity and other attributes of the brain may be determined or inferred by measuring responses of tissue within the brain to light pulses. One technique to measure such responses is time-correlated single-photon counting (TCSPC). Time-correlated single-photon counting detects single photons and measures a time of arrival of the photons with respect to a reference signal (e.g., a light source). By repeating the light pulses, TCSPC may accumulate a sufficient number of photon events to statistically determine a histogram representing the distribution of detected photons. Based on the histogram of photon distribution, the response of tissue to light pulses may be determined in order to study the detected neural activity and/or other attributes of the brain.

A photodetector capable of detecting a single photon (i.e., a single particle of optical energy) is an example of a non-invasive detector that can be used in an optical measurement system to detect neural activity within the brain. An exemplary photodetector is implemented by a semiconductor-based single-photon avalanche diode (SPAD), which is capable of capturing individual photons with very high time-of-arrival resolution (a few tens of picoseconds).

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an exemplary optical measurement system.

FIG. 2 illustrates an exemplary detector architecture.

FIG. 3 illustrates an exemplary timing diagram for performing an optical measurement operation using an optical measurement system.

FIG. 4 illustrates a graph of an exemplary temporal point spread function that may be generated by an optical measurement system in response to a light pulse.

FIG. 5 shows an exemplary non-invasive wearable brain interface system.

FIG. 6 shows an exemplary wearable module assembly.

FIGS. 7A-7B show illustrative configurations that include a wearable assembly in communication with a processing unit.

FIG. 8 illustrates an exemplary implementation of a processing unit.

FIG. 9 shows illustrative signals that may be output by detectors.

FIG. 10 illustrates an exemplary method.

FIG. 11 shows illustrative signals that may be output by detectors.

FIGS. 12-13 show exemplary implementations of a processing unit in which the processing unit is configured to compensate for a motion artifact included in signals output by detectors.

FIG. 14 illustrates an effect of discarding first and second temporal portions of signals that include a motion artifact.

FIG. 15 shows an exemplary configuration in which a wearable assembly includes an inertial measurement unit.

FIG. 16 shows an exemplary implementation of a processing unit in which the processing unit is configured to use movement data output by an inertial measurement unit to determine that intensity changes in first and second signals output by detectors are representative of a motion artifact.

FIGS. 17-22 illustrate embodiments of a wearable device that includes elements of the optical detection systems described herein.

FIG. 23 illustrates an exemplary computing device.

### DETAILED DESCRIPTION

Systems, circuits, and methods for detecting motion artifacts included in signals output by detectors in a wearable optical measurement system are described herein. Such motion artifacts may be caused by movement of a user wearing the wearable optical measurement system. For example, in implementations where a wearable optical measurement system is worn on a head of a user, motion artifacts may be caused by sudden head movement (e.g., when the user relatively quickly turns his or her head, nods his or her head up and down, etc.).

In some examples, the systems, circuits, and methods described herein may compensate for a motion artifact detected in a signal output by a detector of a wearable optical measurement system. For example, the systems, circuits, and methods described herein may remove the motion artifact from the signal, discard a temporal portion of the signal that includes the motion artifact, provide a notification of the motion artifact, and/or perform any other suitable remedial action with respect to the motion artifact. In this manner, the systems, circuits, and methods described herein may minimize or even eliminate any adverse affect that a motion artifact may have on the signal output by the detector and/or one or more measurements and/or operations based on the signal.

These and other advantages and benefits of the present systems, circuits, and methods are described more fully herein.

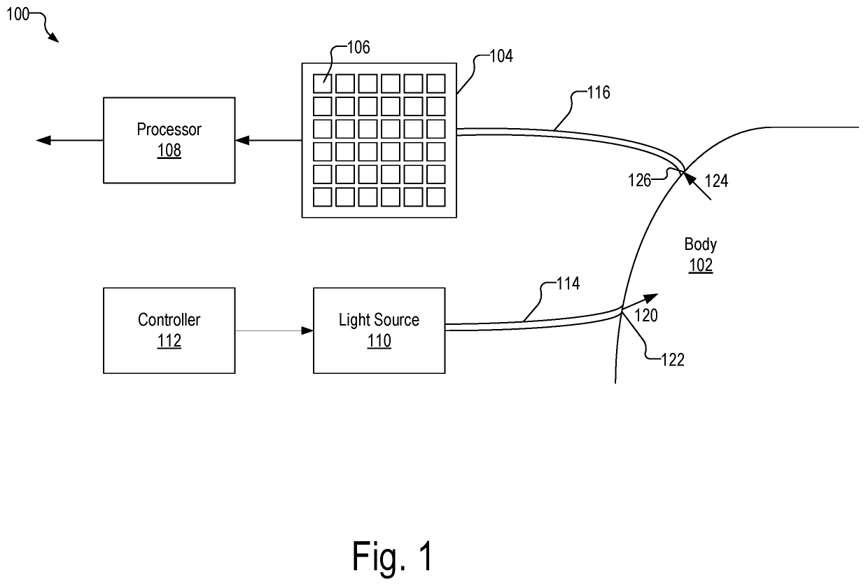

FIG. 1 shows an exemplary optical measurement system 100 configured to perform an optical measurement operation with respect to a body 102. Optical measurement system 100 may, in some examples, be portable and/or wearable by a user. Optical measurement systems that may be used in connection with the embodiments described herein are described more fully in U.S. patent application Ser. No. 17/176,315, filed Feb. 16, 2021; U.S. patent application Ser. No. 17/176,309, filed Feb. 16, 2021; U.S. patent application Ser. No. 17/176,460, filed Feb. 16, 2021; U.S. patent application Ser. No. 17/176,470, filed Feb. 16, 2021; U.S. patent application Ser. No. 17/176,487, filed Feb. 16, 2021; U.S. patent application Ser. No. 17/176,539, filed Feb. 16, 2021; U.S. patent application Ser. No. 17/176,560, filed Feb. 16, 2021; and U.S. patent application Ser. No. 17/176,466, filed Feb. 16, 2021, which applications are incorporated herein by reference in their entirety.

In some examples, optical measurement operations performed by optical measurement system 100 are associated with a time domain-based optical measurement technique. Example time domain-based optical measurement techniques include, but are not limited to, TCSPC, time domain near infrared spectroscopy (TD-NIRS), time domain diffusive correlation spectroscopy (TD-DCS), and time domain Digital Optical Tomography (TD-DOT).

As shown, optical measurement system 100 includes a detector 104 that includes a plurality of individual photodetectors (e.g., photodetector 106), a processor 108 coupled to detector 104, a light source 110, a controller 112, and optical conduits 114 and 116 (e.g., light pipes). However, one or more of these components may not, in certain embodiments, be considered to be a part of optical measurement system 100. For example, in implementations where optical measurement system 100 is wearable by a user, processor 108 and/or controller 112 may in some embodiments be separate from optical measurement system 100 and not configured to be worn by the user.

Detector 104 may include any number of photodetectors 106 as may serve a particular implementation, such as 2ⁿ photodetectors (e.g., 256, 512, . . . , 16384, etc.), where n is an integer greater than or equal to one (e.g., 4, 5, 8, 10, 11, 14, etc.). Photodetectors 106 may be arranged in any suitable manner.

Photodetectors 106 may each be implemented by any suitable circuit configured to detect individual photons of light incident upon photodetectors 106. For example, each photodetector 106 may be implemented by a single photon avalanche diode (SPAD) circuit and/or other circuitry as may serve a particular implementation.

Processor 108 may be implemented by one or more physical processing (e.g., computing) devices. In some examples, processor 108 may execute instructions (e.g., software) configured to perform one or more of the operations described herein.

Light source 110 may be implemented by any suitable component configured to generate and emit light. For example, light source 110 may be implemented by one or more laser diodes, distributed feedback (DFB) lasers, super luminescent diodes (SLDs), light emitting diodes (LEDs), diode-pumped solid-state (DPSS) lasers, super luminescent light emitting diodes (sLEDs), vertical-cavity surface-emitting lasers (VCSELs), titanium sapphire lasers, micro light emitting diodes (mLEDs), and/or any other suitable laser or light source. In some examples, the light emitted by light source 110 is high coherence light (e.g., light that has a coherence length of at least 5 centimeters) at a predetermined center wavelength.

Light source 110 is controlled by controller 112, which may be implemented by any suitable computing device (e.g., processor 108), integrated circuit, and/or combination of hardware and/or software as may serve a particular implementation. In some examples, controller 112 is configured to control light source 110 by turning light source 110 on and off and/or setting an intensity of light generated by light source 110. Controller 112 may be manually operated by a user, or may be programmed to control light source 110 automatically.

Light emitted by light source 110 may travel via an optical conduit 114 (e.g., a light pipe, a light guide, a waveguide, a single-mode optical fiber, and/or or a multi-mode optical fiber) to body 102 of a subject. In cases where optical conduit 114 is implemented by a light guide, the light guide may be spring loaded and/or have a cantilever mechanism to allow for conformably pressing the light guide firmly against body 102.

Body 102 may include any suitable turbid medium. For example, in some implementations, body 102 is a head or any other body part of a human or other animal. Alternatively, body 102 may be a non-living object. For illustrative purposes, it will be assumed in the examples provided herein that body 102 is a human head.

As indicated by arrow 120, the light emitted by light source 110 enters body 102 at a first location 122 on body 102. Accordingly, a distal end of optical conduit 114 may be positioned at (e.g., right above, in physical contact with, or physically attached to) first location 122 (e.g., to a scalp of the subject). In some examples, the light may emerge from optical conduit 114 and spread out to a certain spot size on body 102 to fall under a predetermined safety limit. At least a portion of the light indicated by arrow 120 may be scattered within body 102.

As used herein, “distal” means nearer, along the optical path of the light emitted by light source 110 or the light received by detector 104, to the target (e.g., within body 102) than to light source 110 or detector 104. Thus, the distal end of optical conduit 114 is nearer to body 102 than to light source 110, and the distal end of optical conduit 116 is nearer to body 102 than to detector 104. Additionally, as used herein, “proximal” means nearer, along the optical path of the light emitted by light source 110 or the light received by detector 104, to light source 110 or detector 104 than to body 102. Thus, the proximal end of optical conduit 114 is nearer to light source 110 than to body 102, and the proximal end of optical conduit 116 is nearer to detector 104 than to body 102.

As shown, the distal end of optical conduit 116 (e.g., a light pipe, a light guide, a waveguide, a single-mode optical fiber, and/or a multi-mode optical fiber) is positioned at (e.g., right above, in physical contact with, or physically attached to) output location 126 on body 102. In this manner, optical conduit 116 may collect at least a portion of the scattered light (indicated as light 124) as it exits body 102 at location 126 and carry light 124 to detector 104. Light 124 may pass through one or more lenses and/or other optical elements (not shown) that direct light 124 onto each of the photodetectors 106 included in detector 104.

Photodetectors 106 may be connected in parallel in detector 104. An output of each of photodetectors 106 may be accumulated to generate an accumulated output of detector 104. Processor 108 may receive the accumulated output and determine, based on the accumulated output, a temporal distribution of photons detected by photodetectors 106. Processor 108 may then generate, based on the temporal distribution, a histogram representing a light pulse response of a target (e.g., brain tissue, blood flow, etc.) in body 102. Example embodiments of accumulated outputs are described herein.

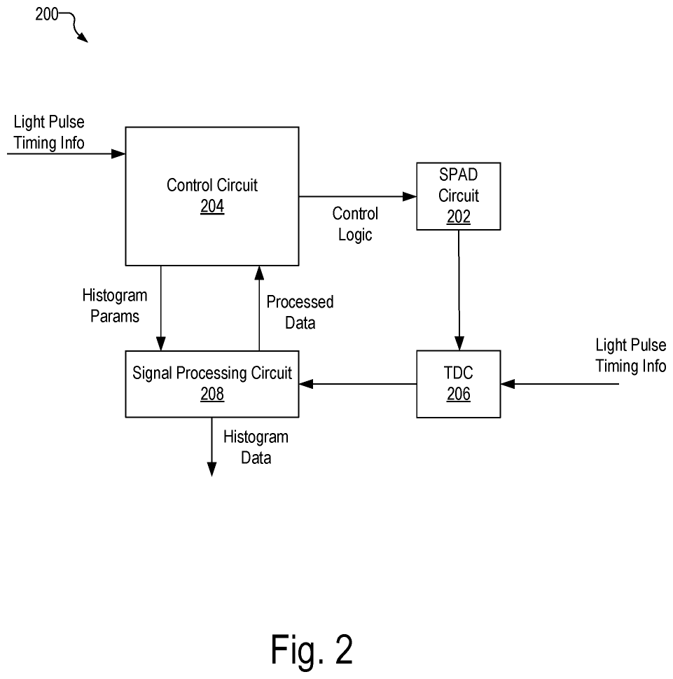

FIG. 2 illustrates an exemplary detector architecture 200 that may be used in accordance with the systems and methods described herein. As shown, architecture 200 includes a SPAD circuit 202 that implements photodetector 106, a control circuit 204, a time-to-digital converter (TDC) 206, and a signal processing circuit 208. Architecture 200 may include additional or alternative components as may serve a particular implementation.

In some examples, SPAD circuit 202 includes a SPAD and a fast gating circuit configured to operate together to detect a photon incident upon the SPAD. As described herein, SPAD circuit 202 may generate an output when SPAD circuit 202 detects a photon.

The fast gating circuit included in SPAD circuit 202 may be implemented in any suitable manner. For example, the fast gating circuit may include a capacitor that is pre-charged with a bias voltage before a command is provided to arm the SPAD. Gating the SPAD with a capacitor instead of with an active voltage source, such as is done in some conventional SPAD architectures, has a number of advantages and benefits. For example, a SPAD that is gated with a capacitor may be armed practically instantaneously compared to a SPAD that is gated with an active voltage source. This is because the capacitor is already charged with the bias voltage when a command is provided to arm the SPAD. This is described more fully in U.S. Pat. Nos. 10,158,038 and 10,424,683, which are incorporated herein by reference in their respective entireties.

In some alternative configurations, SPAD circuit 202 does not include a fast gating circuit. In these configurations, the SPAD included in SPAD circuit 202 may be gated in any suitable manner or be configured to operate in a free running mode with passive quenching.

Control circuit 204 may be implemented by an application specific integrated circuit (ASIC) or any other suitable circuit configured to control an operation of various components within SPAD circuit 202. For example, control circuit 204 may output control logic that puts the SPAD included in SPAD circuit 202 in either an armed or a disarmed state.

In some examples, control circuit 204 may control a gate delay, which specifies a predetermined amount of time control circuit 204 is to wait after an occurrence of a light pulse (e.g., a laser pulse) to put the SPAD in the armed state. To this end, control circuit 204 may receive light pulse timing information, which indicates a time at which a light pulse occurs (e.g., a time at which the light pulse is applied to body 102). Control circuit 204 may also control a programmable gate width, which specifies how long the SPAD is kept in the armed state before being disarmed.

Control circuit 204 is further configured to control signal processing circuit 208. For example, control circuit 204 may provide histogram parameters (e.g., time bins, number of light pulses, type of histogram, etc.) to signal processing circuit 208. Signal processing circuit 208 may generate histogram data in accordance with the histogram parameters. In some examples, control circuit 204 is at least partially implemented by controller 112.

TDC 206 is configured to measure a time difference between an occurrence of an output pulse generated by SPAD circuit 202 and an occurrence of a light pulse. To this end, TDC 206 may also receive the same light pulse timing information that control circuit 204 receives. TDC 206 may be implemented by any suitable circuitry as may serve a particular implementation.

Signal processing circuit 208 is configured to perform one or more signal processing operations on data output by TDC 206. For example, signal processing circuit 208 may generate histogram data based on the data output by TDC 206 and in accordance with histogram parameters provided by control circuit 204. To illustrate, signal processing circuit 208 may generate, store, transmit, compress, analyze, decode, and/or otherwise process histograms based on the data output by TDC 206. In some examples, signal processing circuit 208 may provide processed data to control circuit 204, which may use the processed data in any suitable manner. In some examples, signal processing circuit 208 is at least partially implemented by processor 108.

In some examples, each photodetector 106 (e.g., SPAD circuit 202) may have a dedicated TDC 206 associated therewith. For example, for an array of N photodetectors 106, there may be a corresponding array of N TDCs 206. Alternatively, a single TDC 206 may be associated with multiple photodetectors 106. Likewise, a single control circuit 204 and a single signal processing circuit 208 may be provided for a one or more photodetectors 106 and/or TDCs 206.

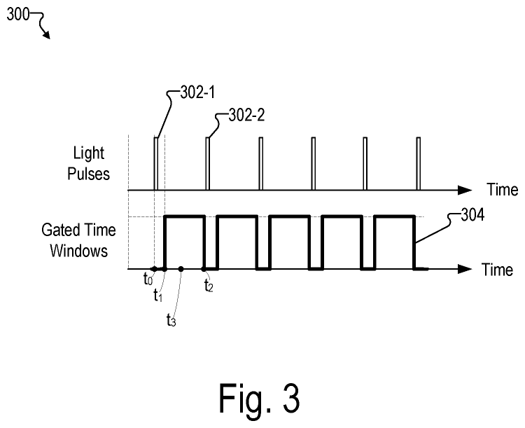

FIG. 3 illustrates an exemplary timing diagram 300 for performing an optical measurement operation using optical measurement system 100. Optical measurement system 100 may be configured to perform the optical measurement operation by directing light pulses (e.g., laser pulses) toward a target within a body (e.g., body 102). The light pulses may be short (e.g., 10-2000 picoseconds (ps)) and repeated at a high frequency (e.g., between 100,000 hertz (Hz) and 100 megahertz (MHz)). The light pulses may be scattered by the target and then detected by optical measurement system 100. Optical measurement system 100 may measure a time relative to the light pulse for each detected photon. By counting the number of photons detected at each time relative to each light pulse repeated over a plurality of light pulses, optical measurement system 100 may generate a histogram that represents a light pulse response of the target (e.g., a temporal point spread function (TPSF)). The terms histogram and TPSF are used interchangeably herein to refer to a light pulse response of a target.

For example, timing diagram 300 shows a sequence of light pulses 302 (e.g., light pulses 302-1 and 302-2) that may be applied to the target (e.g., tissue within a brain of a user, blood flow, a fluorescent material used as a probe in a body of a user, etc.). Timing diagram 300 also shows a pulse wave 304 representing predetermined gated time windows (also referred as gated time periods) during which photodetectors 106 are gated ON to detect photons. Referring to light pulse 302-1, light pulse 302-1 is applied at a time t₀. At a time t₁, a first instance of the predetermined gated time window begins. Photodetectors 106 may be armed at time t₁, enabling photodetectors 106 to detect photons scattered by the target during the predetermined gated time window. In this example, time t₁ is set to be at a certain time after time t₀, which may minimize photons detected directly from the laser pulse, before the laser pulse reaches the target. However, in some alternative examples, time t₁ is set to be equal to time t₀.

At a time t₂, the predetermined gated time window ends. In some examples, photodetectors 106 may be disarmed at time t₂. In other examples, photodetectors 106 may be reset (e.g., disarmed and re-armed) at time t₂ or at a time subsequent to time t₂. During the predetermined gated time window, photodetectors 106 may detect photons scattered by the target. Photodetectors 106 may be configured to remain armed during the predetermined gated time window such that photodetectors 106 maintain an output upon detecting a photon during the predetermined gated time window. For example, a photodetector 106 may detect a photon at a time t₃, which is during the predetermined gated time window between times t₁ and t₂. The photodetector 106 may be configured to provide an output indicating that the photodetector 106 has detected a photon. The photodetector 106 may be configured to continue providing the output until time t₂, when the photodetector may be disarmed and/or reset. Optical measurement system 100 may generate an accumulated output from the plurality of photodetectors. Optical measurement system 100 may sample the accumulated output to determine times at which photons are detected by photodetectors 106 to generate a TPSF.

As mentioned, in some alternative examples, photodetector 106 may be configured to operate in a free-running mode such that photodetector 106 is not actively armed and disarmed (e.g., at the end of each predetermined gated time window represented by pulse wave 304). In contrast, while operating in the free-running mode, photodetector 106 may be configured to reset within a configurable time period after an occurrence of a photon detection event (i.e., after photodetector 106 detects a photon) and immediately begin detecting new photons. However, only photons detected within a desired time window (e.g., during each gated time window represented by pulse wave 304) may be included in the TPSF.

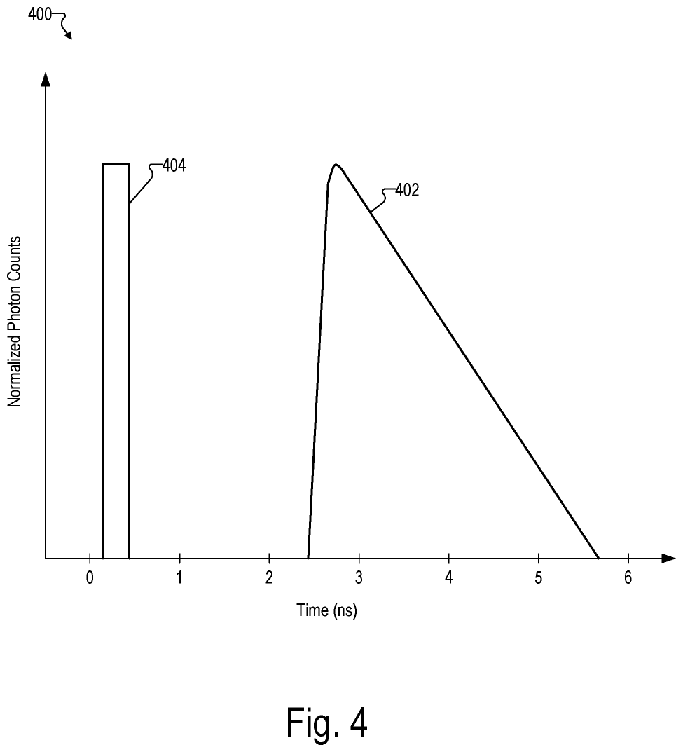

FIG. 4 illustrates a graph 400 of an exemplary TPSF 402 that may be generated by optical measurement system 100 in response to a light pulse 404 (which, in practice, represents a plurality of light pulses). Graph 400 shows a normalized count of photons on a y-axis and time bins on an x-axis. As shown, TPSF 402 is delayed with respect to a temporal occurrence of light pulse 404. In some examples, the number of photons detected in each time bin subsequent to each occurrence of light pulse 404 may be aggregated (e.g., integrated) to generate TPSF 402. TPSF 402 may be analyzed and/or processed in any suitable manner to determine or infer detected neural activity.

Optical measurement system 100 may be implemented by or included in any suitable device. For example, optical measurement system 100 may be included, in whole or in part, in a non-invasive wearable device (e.g., a headpiece) that a user may wear to perform one or more diagnostic, imaging, analytical, and/or consumer-related operations. The non-invasive wearable device may be placed on a user's head or other part of the user to detect neural activity. In some examples, such neural activity may be used to make behavioral and mental state analysis, awareness and predictions for the user.

Mental state described herein refers to the measured neural activity related to physiological brain states and/or mental brain states, e.g., joy, excitement, relaxation, surprise, fear, stress, anxiety, sadness, anger, disgust, contempt, contentment, calmness, focus, attention, approval, creativity, positive or negative reflections/attitude on experiences or the use of objects, etc. Further details on the methods and systems related to a predicted brain state, behavior, preferences, or attitude of the user, and the creation, training, and use of neuromes can be found in U.S. Provisional Patent Application No. 63/047,991, filed Jul. 3, 2020. Exemplary measurement systems and methods using biofeedback for awareness and modulation of mental state are described in more detail in U.S. patent application Ser. No. 16/364,338, filed Mar. 26, 2019, published as US2020/0196932A1. Exemplary measurement systems and methods used for detecting and modulating the mental state of a user using entertainment selections, e.g., music, film/video, are described in more detail in U.S. patent application Ser. No. 16/835,972, filed Mar. 31, 2020, published as US2020/0315510A1. Exemplary measurement systems and methods used for detecting and modulating the mental state of a user using product formulation from, e.g., beverages, food, selective food/drink ingredients, fragrances, and assessment based on product-elicited brain state measurements are described in more detail in U.S. patent application Ser. No. 16/853,614, filed Apr. 20, 2020, published as US2020/0337624A1. Exemplary measurement systems and methods used for detecting and modulating the mental state of a user through awareness of priming effects are described in more detail in U.S. patent application Ser. No. 16/885,596, filed May 28, 2020, published as US2020/0390358A1. These applications and corresponding U.S. publications are incorporated herein by reference in their entirety.

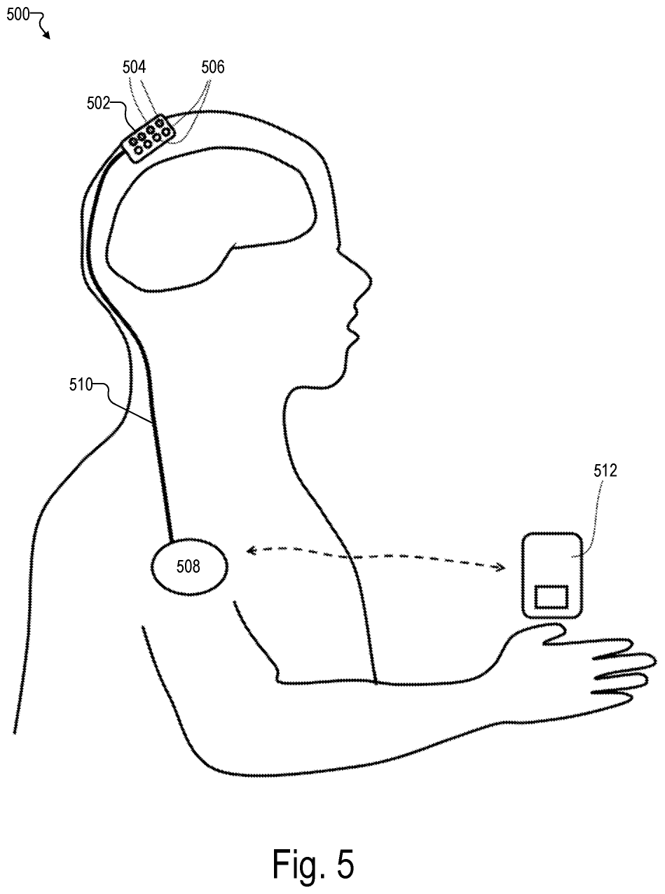

FIG. 5 shows an exemplary non-invasive wearable brain interface system 500 (“brain interface system 500”) that implements optical measurement system 100 (shown in FIG. 1). As shown, brain interface system 500 includes a head-mountable component 502 configured to be attached to a user's head. Head-mountable component 502 may be implemented by a cap shape that is worn on a head of a user. Alternative implementations of head-mountable component 502 include helmets, beanies, headbands, other hat shapes, or other forms conformable to be worn on a user's head, etc. Head-mountable component 502 may be made out of any suitable cloth, soft polymer, plastic, hard shell, and/or any other suitable material as may serve a particular implementation. Examples of headgears used with wearable brain interface systems are described more fully in U.S. Pat. No. 10,340,408, incorporated herein by reference in its entirety.

Head-mountable component 502 includes a plurality of detectors 504, which may implement or be similar to detector 104, and a plurality of light sources 506, which may be implemented by or be similar to light source 110. It will be recognized that in some alternative embodiments, head-mountable component 502 may include a single detector 504 and/or a single light source 506.

Brain interface system 500 may be used for controlling an optical path to the brain and for transforming photodetector measurements into an intensity value that represents an optical property of a target within the brain. Brain interface system 500 allows optical detection of deep anatomical locations beyond skin and bone (e.g., skull) by extracting data from photons originating from light source 506 and emitted to a target location within the user's brain, in contrast to conventional imaging systems and methods (e.g., optical coherence tomography (OCT)), which only image superficial tissue structures or through optically transparent structures.

Brain interface system 500 may further include a processor 508 configured to communicate with (e.g., control and/or receive signals from) detectors 504 and light sources 506 by way of a communication link 510. Communication link 510 may include any suitable wired and/or wireless communication link. Processor 508 may include any suitable housing and may be located on the user's scalp, neck, shoulders, chest, or arm, as may be desirable. In some variations, processor 508 may be integrated in the same assembly housing as detectors 504 and light sources 506.

As shown, brain interface system 500 may optionally include a remote processor 512 in communication with processor 508. For example, remote processor 512 may store measured data from detectors 504 and/or processor 508 from previous detection sessions and/or from multiple brain interface systems (not shown). Power for detectors 504, light sources 506, and/or processor 508 may be provided via a wearable battery (not shown). In some examples, processor 508 and the battery may be enclosed in a single housing, and wires carrying power signals from processor 508 and the battery may extend to detectors 504 and light sources 506. Alternatively, power may be provided wirelessly (e.g., by induction).

In some alternative embodiments, head mountable component 502 does not include individual light sources. Instead, a light source configured to generate the light that is detected by detector 504 may be included elsewhere in brain interface system 500. For example, a light source may be included in processor 508 and coupled to head mountable component 502 through optical connections.

Optical measurement system 100 may alternatively be included in a non-wearable device (e.g., a medical device and/or consumer device that is placed near the head or other body part of a user to perform one or more diagnostic, imaging, and/or consumer-related operations). Optical measurement system 100 may alternatively be included in a sub-assembly enclosure of a wearable invasive device (e.g., an implantable medical device for brain recording and imaging).

Optical measurement system 100 may be modular in that one or more components of optical measurement system 100 may be removed, changed out, or otherwise modified as may serve a particular implementation. Additionally or alternatively, optical measurement system 100 may be modular such that one or more components of optical measurement system 100 may be housed in a separate housing (e.g., module) and/or may be movable relative to other components. Exemplary modular multimodal measurement systems are described in more detail in U.S. Provisional patent application Ser. No. 17/176,460, filed Feb. 16, 2021, U.S. Provisional patent application Ser. No. 17/176,470, filed Feb. 16, 2021, U.S. Provisional patent application Ser. No. 17/176,487, filed Feb. 16, 2021, U.S. Provisional Patent Application No. 63/038,481, filed Feb. 16, 2021, and U.S. Provisional patent application Ser. No. 17/176,560, filed Feb. 16, 2021, which applications are incorporated herein by reference in their respective entireties.

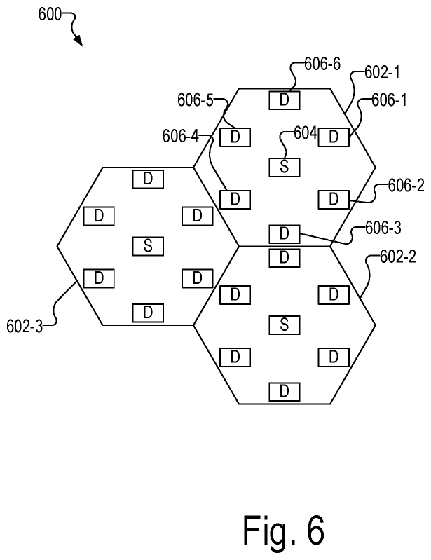

To illustrate, FIG. 6 shows an exemplary wearable module assembly 600 (“assembly 600”) that implements one or more of the optical measurement features described herein. Assembly 600 may be worn on the head or any other suitable body part of the user. As shown, assembly 600 may include a plurality of modules 602 (e.g., modules 602-1 through 602-3). While three modules 602 are shown to be included in assembly 600 in FIG. 6, in alternative configurations, any number of modules 602 (e.g., a single module up to sixteen or more modules) may be included in assembly 600. Moreover, while modules 602 are shown to be adjacent to and touching one another, modules 602 may alternatively be spaced apart from one another (e.g., in implementations where modules 602 are configured to be inserted into individual slots or cutouts of the headgear). Moreover, while modules 602 are shown to have a hexagonal shape, modules 602 may alternatively have any other suitable geometry (e.g., in the shape of a pentagon, octagon, square, rectangular, circular, triangular, free-form, etc.). Assembly 600 may conform to three-dimensional surface geometries, such as a user's head. Exemplary wearable module assemblies comprising a plurality of wearable modules are described in more detail in U.S. Provisional Patent Application No. 62/992,550, filed Mar. 20, 2020, and U.S. Provisional Patent Application No. 63/038,458, filed Jun. 12, 2020, which applications are incorporated herein by reference in their respective entireties.

Each module 602 includes a source 604 and a plurality of detectors 606 (e.g., detectors 606-1 through 606-6). Source 604 may be implemented by one or more light sources similar to light source 110. Each detector 606 may implement or be similar to detector 104 and may include a plurality of photodetectors (e.g., SPADs) as well as other circuitry (e.g., TDCs). As shown, detectors 606 are arranged around and substantially equidistant from source 604. In other words, the spacing between a light source (i.e., a distal end portion of a light source optical conduit) and the detectors (i.e., distal end portions of optical conduits for each detector) are maintained at the same fixed distance on each module to ensure homogeneous coverage over specific areas and to facilitate processing of the detected signals. The fixed spacing also provides consistent spatial (lateral and depth) resolution across the target area of interest, e.g., brain tissue. Moreover, maintaining a known distance between the light emitter and the detector allows subsequent processing of the detected signals to infer spatial (e.g., depth localization, inverse modeling) information about the detected signals. Detectors 606 may be alternatively disposed as may serve a particular implementation.

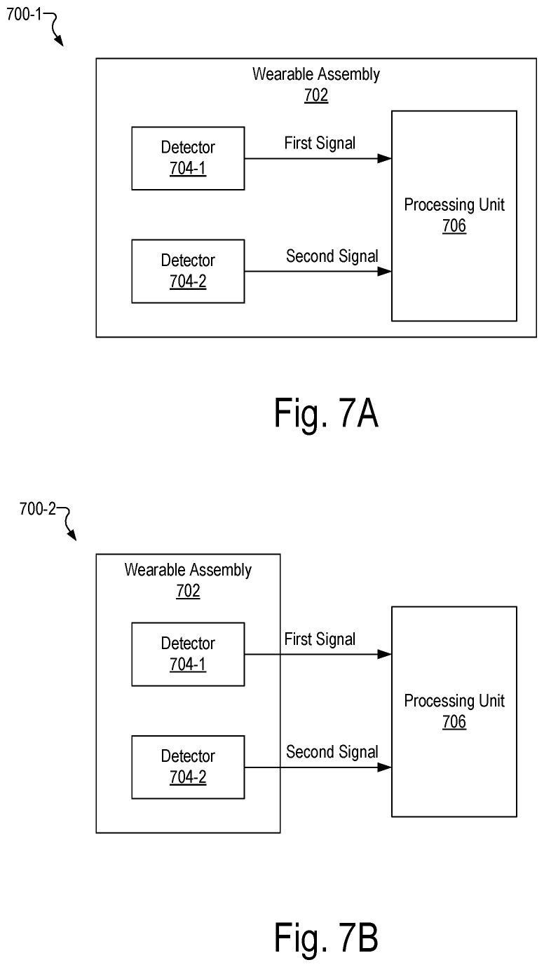

FIGS. 7A-7B show illustrative configurations 700-1 and 700-2 in accordance with the principles described herein. Each configuration 700 includes a wearable assembly 702 having a plurality of detectors 704 (e.g., detector 704-1 and 704-2) in communication with a processing unit 706. In configuration 700-1, processing unit 706 is included in wearable assembly 702, while in configuration 700-2, processing unit 706 is not included in wearable assembly 702. Either configuration 700-1 or 700-2 may be used in accordance with the systems, circuits, and methods described herein.

Wearable assembly 702 may be implemented by any of the wearable devices, wearable module assemblies, and/or wearable units described herein. For example, wearable assembly 702 may be implemented by a wearable device configured to be worn on a user's head. Wearable assembly 702 may additionally or alternatively be configured to be worn on any other part of a user's body.

Detectors 704 may be implemented by any of the detectors described herein (e.g., any of the detectors 606 shown in FIG. 6). While two detectors 704 are shown to be included in wearable assembly 702, it will be recognized that any number of detectors 704 may be included in wearable assembly 702.

In some examples, detectors 704 are each located on a particular module (e.g., module 602-1) included in wearable assembly 702. In alternative implementations, detectors 704 are located on separate modules (e.g., detector 704-1 may be located on module 606-1 and detector 704-2 may be located on module 606-2).

Detector 704-1 is configured to detect a first set of photon arrival times (e.g., timestamp symbols representative of times at which photons are detected by a photodetector included in detector 704-1) and output a first signal representative of the first set of photon arrival times. Likewise, detector 704-2 is configured to detect a second set of photon arrival times and output a second signal representative of the second set of photon arrival times. The first and second signals may be used to generate one or more histograms, as described herein.

As mentioned, in configuration 700-2, processing unit 706 is not included in wearable assembly 702. To illustrate, processing unit 706 may be included in a wearable device separate from wearable assembly 702. For example, processing unit 706 may be included in a wearable device configured to be worn off the head while wearable assembly 702 is worn on the head. In these examples, one or more communication interfaces (e.g., cables, wireless interfaces, etc.) may be used to facilitate wearable assembly 702 and the separate wearable device.

Additionally or alternatively, in configuration 700-2, processing unit 706 may be remote from the user (i.e., not worn by the user). For example, processing unit 706 may be implemented by a stand-alone computing device communicatively coupled to wearable assembly 702 by way of one or more communication interfaces (e.g., cables, wireless interfaces, etc.).

Processing unit 706 may be implemented by processor 108, controller 112, control circuit 204, and/or any other suitable processing and/or computing device or circuit.

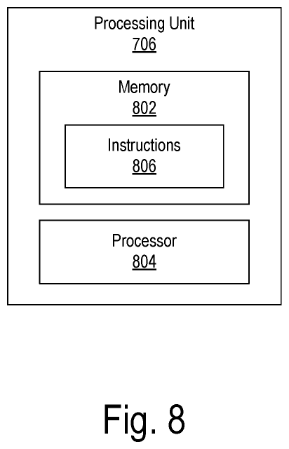

For example, FIG. 8 illustrates an exemplary implementation of processing unit 706 in which processing unit 706 includes a memory 802 and a processor 804 configured to be selectively and communicatively coupled to one another. In some examples, memory 802 and processor 804 may be distributed between multiple devices and/or multiple locations as may serve a particular implementation.

Memory 802 may be implemented by any suitable non-transitory computer-readable medium and/or non-transitory processor-readable medium, such as any combination of non-volatile storage media and/or volatile storage media. Exemplary non-volatile storage media include, but are not limited to, read-only memory, flash memory, a solid-state drive, a magnetic storage device (e.g., a hard drive), ferroelectric random-access memory (“RAM”), and an optical disc. Exemplary volatile storage media include, but are not limited to, RAM (e.g., dynamic RAM).

Memory 802 may maintain (e.g., store) executable data used by processor 804 to perform one or more of the operations described herein. For example, memory 802 may store instructions 806 that may be executed by processor 804 to perform any of the operations described herein. Instructions 806 may be implemented by any suitable application, program (e.g., sound processing program), software, code, and/or other executable data instance. Memory 802 may also maintain any data received, generated, managed, used, and/or transmitted by processor 804.

Processor 804 may be configured to perform (e.g., execute instructions 806 stored in memory 802 to perform) various operations described herein. For example, processor 804 may be configured to perform any of the operations described herein as being performed by processing unit 706.

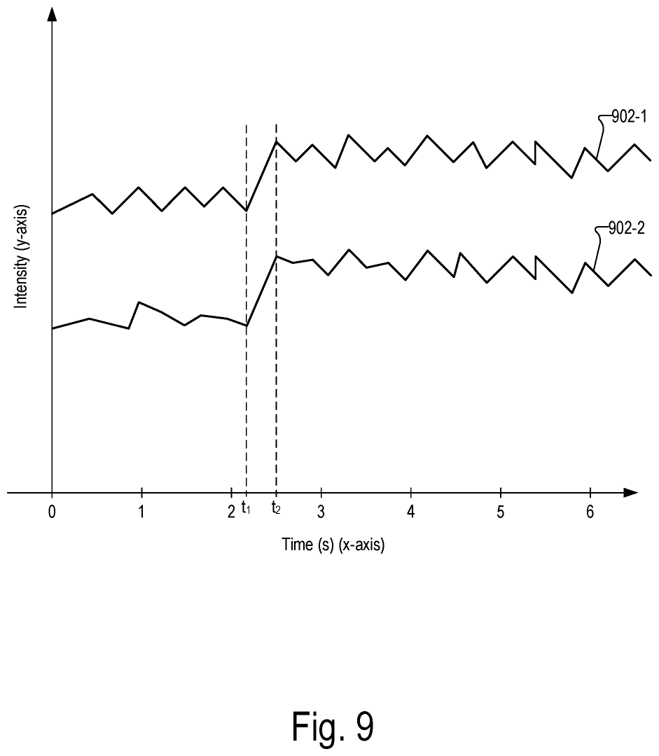

FIG. 9 shows illustrative signals that may be output by detectors 704. In particular, FIG. 9 shows a first signal 902-1 that may be output by detector 704-1 and a second signal 902-2 that may be output by detector 704-2. As shown, signals 902 may each vary in intensity (y-axis) over time (x-axis). In this example, the time scale of the x-axis is in seconds (which is relatively much longer than the time scale for a particular histogram that may be generated based on signals 902-1). Hence, the time scale shown in FIG. 9 indicates how signal intensity may vary over time as the user wears wearable assembly 702 throughout the course of a wearing session (e.g., an hour, a day, etc.), for example. The intensity of signal 902-1 is shown to be generally higher than the intensity of signal 902-2 for illustrative purposes only. It will be recognized that signals 902 may each have any suitable intensity at any given time.

In some situations, a user wearing wearable assembly 702 may make a movement that causes a motion artifact to appear in signals 902. The movement may include the user moving a body part to which wearable assembly 702 is attached (e.g., by turning or nodding his or her head), making a sudden movement (e.g., by standing up, sitting down, lying down, etc.), making a chewing motion, tilting his or her head back to drink, falling, running, and/or any other movement.

A motion artifact may be manifest in signals 902 in any suitable manner. For example, a motion artifact may cause the intensity of signals 902 to change by at least a threshold amount during a particular time period. To illustrate, FIG. 9 shows that both signals 902 include a motion artifact that causes both signals 902 to change in intensity by a relatively large amount between times t₁ and t₂.

Because detectors 704 are attached to the same wearable assembly 702 (e.g., rigidly attached to the same module in wearable assembly 702), detectors 704 may move in a coordinated manner in response to movement by the user. As such, as shown in FIG. 9, the intensity changes in signals 902 caused by the motion artifact are time correlated. As used herein, intensity changes in two signals (e.g., signals 902-1 and 902-2) are time correlated if they occur during a same time period (e.g., between times t₁ and t₂, as shown in FIG. 9) or within a predetermined offset amount of time as may serve a particular implementation. For example, due to differences in physical location of detectors 704, an intensity change in signal 902-1 may be slightly offset in time compared to the intensity change in signal 902-2, but still be time correlated. Any suitable signal processing heuristic and/or statistical model may be used to determine whether intensity changes in signals 902 are time correlated.

Hence, processing unit 706 may identify a motion artifact by detecting time correlated intensity changes in signals output by multiple detectors. Such time correlated intensity changes may be detected between two detectors (e.g., detectors 704-1 and 704-2) or any other suitable combination of multiple detectors (e.g., all the detectors on a particular module 602, as shown in FIG. 6) as may serve a particular implementation).

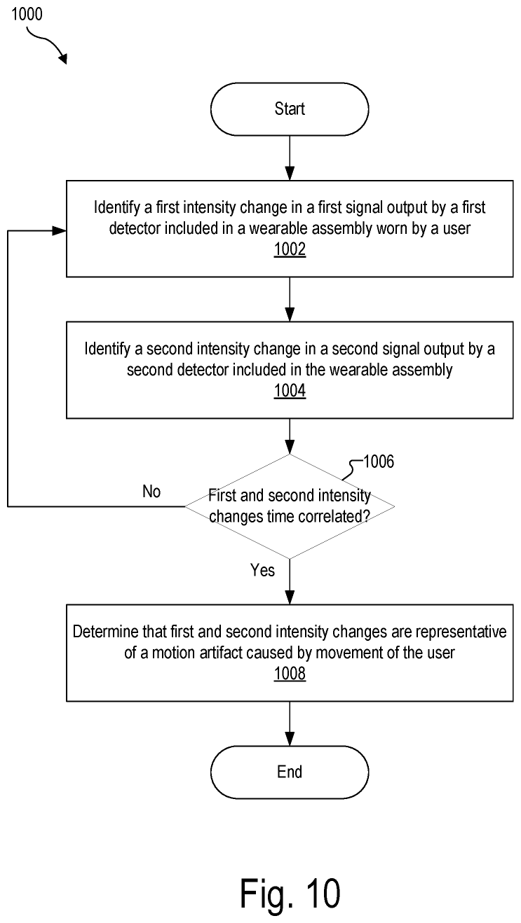

To illustrate, FIG. 10 illustrates an exemplary method 1000 that may be performed by processing unit 706 to identify motion artifact in signals output by first and second detectors (e.g., detectors 704-1 and 704-2). While FIG. 10 illustrates exemplary operations according to one embodiment, other embodiments may omit, add to, reorder, and/or modify any of the operations shown in FIG. 10.

At operation 1002, processing unit 706 identifies a first intensity change in a first signal (e.g., signal 902-1) output by a first detector (e.g., detector 704-1) included in a wearable assembly (e.g., wearable assembly 702) worn by a user. In some examples, this identification is performed by detecting that the intensity of the first signal changes by at least a threshold amount (this is because relatively small variations in intensity are to be expected and may not be caused by motion artifacts) during a particular amount of time (e.g., in less than a second or any other suitable amount of time).

At operation 1004, processing unit 706 identifies a second intensity change in a second signal (e.g., signal 902-2) output by a second detector (e.g., detector 704-2) included in the wearable assembly. Again, this identification may be performed by detecting that the intensity of the second signal changes by at least a threshold amount during a particular amount of time.

At decision 1006, processing unit 706 determines whether the first and second intensity changes are time correlated. If the first and second intensity changes are time correlated (Yes; decision 1006), processing unit 706 determines, at operation 1008, that the first and second intensity changes are representative of a motion artifact caused by movement of the user. If the first and second intensity changes are not time correlated (No; decision 1006), processing unit 706 continues monitoring for intensity changes in the first and second signals.

In some examples, processing unit 706 may determine whether the first and second intensity changes are time correlated in response to determining that the and second intensity changes are both greater than a threshold amount. In this manner, relatively small intensity changes not may not be processed to determine whether they are time correlated.

Additionally or alternatively, processing unit 706 may determine that the first and second intensity changes are time correlated by determining that the first and second intensity changes are time correlated for at least a threshold amount of time. This threshold amount of time may be based on one or more settings or characteristics of the optical measurement system and/or on user input. For example, with respect to the example provided in FIG. 9, processing unit 706 may determine that a time duration defined by t₂-t₁ is greater than a threshold amount of time. In response, processing unit 706 may determine that the intensity changes that occur during the time duration defined by t₂-t₁ are time correlated.

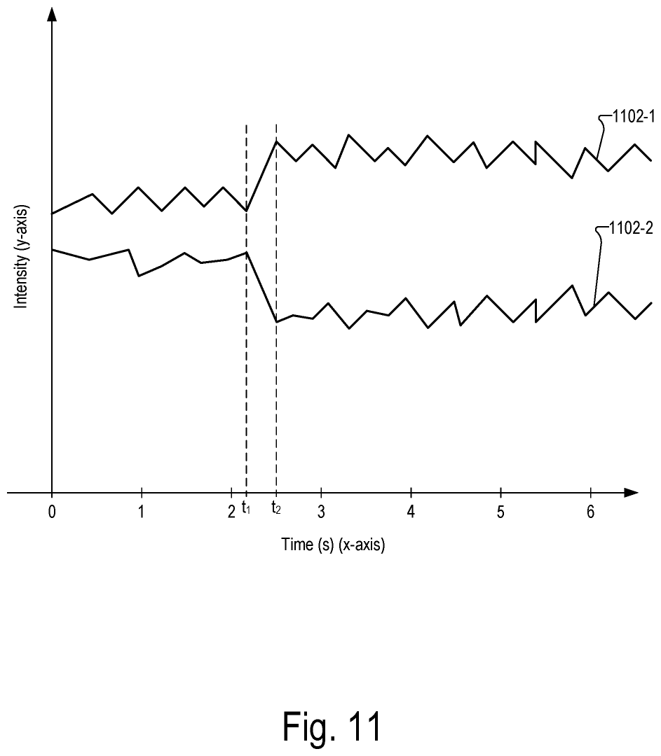

In some examples, intensity changes in signals output by multiple detectors that occur because of motion artifacts may be equal in direction. For example, in the example of FIG. 9, the intensity changes in signals 902 caused by the motion artifact are both positive (i.e., the intensities of both signals 902 increase in amplitude due to the motion artifact). Alternatively, a motion artifact may affect the intensities of signals output by multiple detectors in opposite manners. To illustrate, FIG. 11 shows illustrative signals 1102-1 and 1102-2 that may be output by detectors 704-1 and 704-2, respectively. As shown, a motion artifact may cause signal 1102-1 to increase in intensity during the time period defined by t₁ and t₂, while the same motion artifact may cause signal 1102-2 to decrease in intensity during the same time period.

As such, in some examples, to determine whether intensity changes of multiple signals are time correlated, processing unit 706 may first take the derivative of each signal. Processing unit 706 may then compare amplitude changes of the derivative signals to determine whether intensity changes occur and whether such intensity changes are time correlated.

Processing unit 706 may be configured to compensate for a motion artifact included in one or more signals output by detectors 704. Various ways in which this compensation may be performed will now be described.

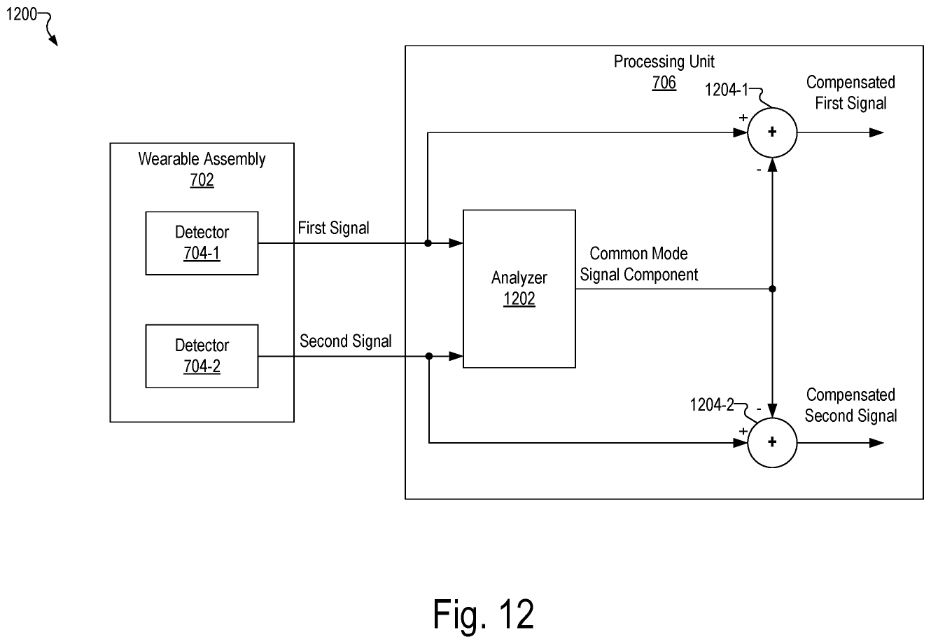

FIG. 12 shows an exemplary implementation 1200 of processing unit 706 in which processing unit 706 is configured to compensate for a motion artifact included in signals (e.g., signals 902 or signals 1102) output by detectors 704. The various components shown in FIG. 12 as being included in processing unit 706 may be implemented by suitable combination of hardware, circuits, and/or software.

As shown, an analyzer 1202 included in processing unit 706 may receive the first and second signals output by detectors 704-1 and 704-2, respectively. Analyzer 1202 is configured to analyze the first and second signals to determine a common mode signal component included in both the first and second signals. This analysis may be performed in any suitable manner. For example, analyzer 1202 may compare one or more temporal and/or spectral characteristics of the first and second signals and generate, based on the comparison, the common mode signal component. As detectors 704 are affected similarly by the motion artifact, the common mode signal component may include the motion artifact.

Hence, processing unit 706 may compensate for the motion artifact by subtracting the common mode signal component from both the first and second signals.

For example, to subtract the common mode signal component from the first signal, processing unit 706 may input the common mode signal component into a summation block 1204-1, which also receives the first signal component. Summation block 1204-1 may subtract the common mode signal component from the first signal and output a compensated first signal. This compensated first signal may accordingly not include (or only include a negligible amount of) the motion artifact.

Likewise, to subtract the common mode signal component from the second signal, processing unit 706 may input the common mode signal component into a summation block 1204-2, which also receives the second signal component. Summation block 1204-2 may subtract the common mode signal component from the second signal and output a compensated second signal. This compensated second signal may accordingly not include (or only include a negligible amount of) the motion artifact.

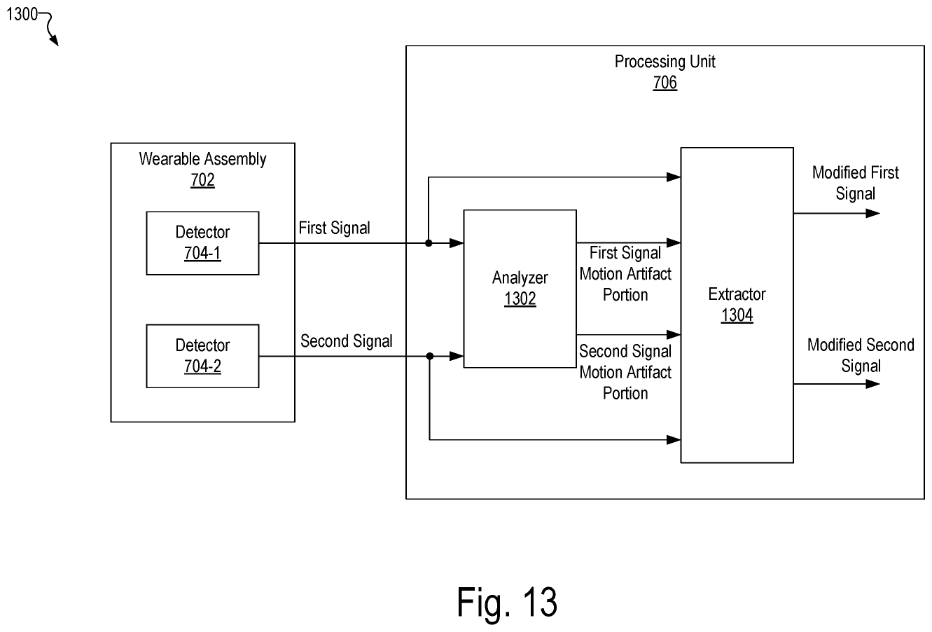

FIG. 13 shows another exemplary implementation 1300 of processing unit 706 in which processing unit 706 is configured to compensate for a motion artifact included in signals (e.g., signals 902 or signals 1102) output by detectors 704. The various components shown in FIG. 13 as being included in processing unit 706 may be implemented by suitable combination of hardware, circuits, and/or software.

As shown, an analyzer 1302 included in processing unit 706 may receive the first and second signals output by detectors 704-1 and 704-2, respectively. Analyzer 1302 is configured to analyze the first signal to identify a first temporal portion, within the first signal, that includes the motion artifact. Likewise, analyzer 1302 is configured to analyze the second signal to identify a second temporal portion, within the second signal, that includes the motion artifact. As shown, analyzer 1302 may output “first signal motion artifact portion” data that identifies the first temporal portion and “second signal motion artifact portion” data that identifies the second temporal portion.

To illustrate, with respect to signals 902, the first temporal portion identified by analyzer 1302 may include the portion of signal 902-1 between times t₁ and t₂. Likewise, the second temporal portion identified by analyzer 1302 may include the portion of signal 902-2 between times t₁ and t₂.

In some examples, processing unit 706 may compensate for the motion artifact by discarding the first temporal portion from the first signal and/or discarding the second temporal portion from the second signal. To illustrate, as shown in FIG. 13, processing unit 706 may include an extractor 1304 configured to receive the first signal, the second signal, the first signal motion artifact portion data, and the second signal motion artifact portion data. Extractor 1304 may be configured to discard, based on the first signal motion artifact portion data, the first temporal portion from the first signal. Likewise, extractor 1304 may be configured to discard, based on the second signal motion artifact portion data, the second temporal portion from the second signal. Extractor 1304 may accordingly output a modified first signal that does not include the first temporal portion and a modified second signal that does not include the second temporal portion.

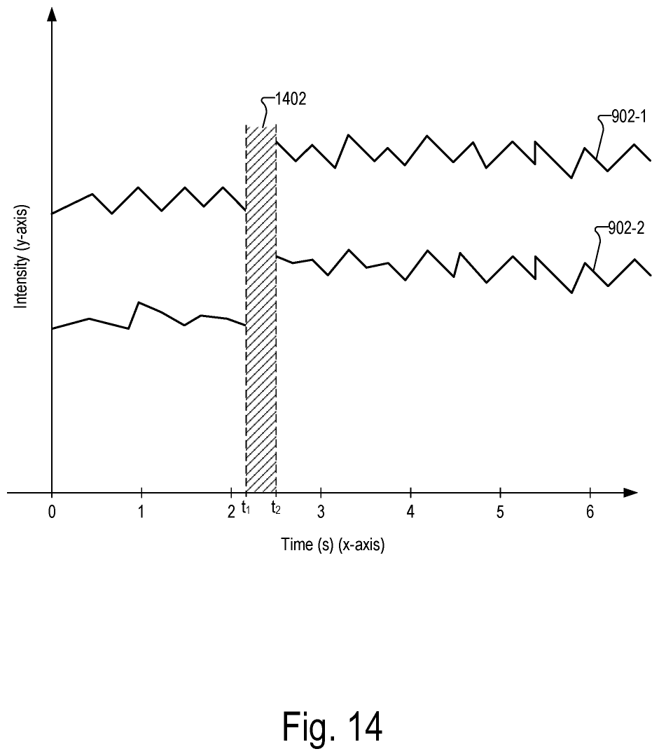

FIG. 14 illustrates an effect of discarding the first and second temporal portions of signals 902 that include the motion artifact. As illustrated by a hatched-filled in block 1402 that blocks out the portions of signals 902 between times t₁ and t₂, the first and second temporal portions are no longer included in signals 902.

In some examples, wearable assembly 702 may include a motion sensor that may be used by processing unit 706 to confirm that a signal artifact is due to motion.

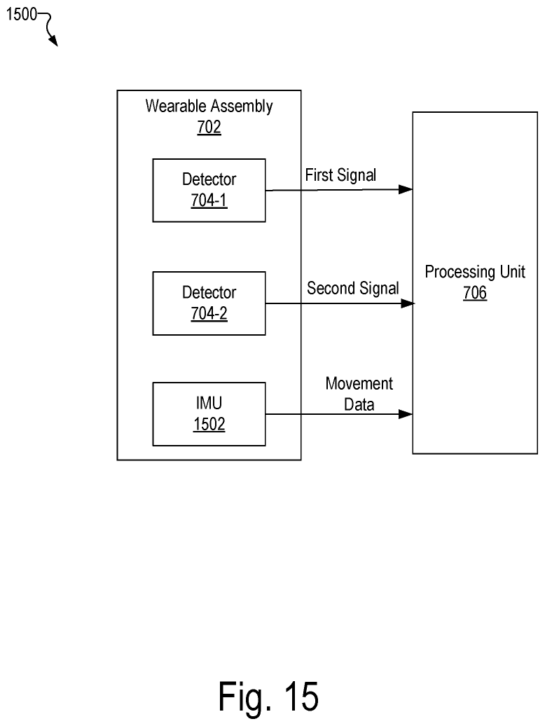

To illustrate, FIG. 15 shows an exemplary configuration 1500 in which wearable assembly 702 includes an inertial measurement unit (IMU) 1502. IMU 1502 may include one or more accelerometers, gyroscopes, magnetometers, and/or other motion sensors as may serve a particular implementation. IMU 1502 may be included in wearable assembly 702 in any suitable manner. For example, IMU 1502 may be located on a dedicated module, located on a module that also includes detectors 704, and/or in any other suitable location. In some alternative implementations, IMU 1502 is not included in wearable assembly 702 and is instead worn in an alternative manner by the user.

As shown, IMU 1502 may be configured to output movement data associated with wearable assembly 702. The movement data may be constantly output (e.g., streamed) by IMU 1502 and/or output in response to an occurrence of a predetermined event and/or a condition that is satisfied.

The movement data may be indicative of a movement of the wearable assembly 702 (and, hence, a user of wearable assembly 702). The movement data may be in any suitable format and may represent a characteristic (e.g., distance, velocity, and/or acceleration) of the movement. As described herein, the movement data may therefore be used by processing unit 706 to classify movement made by the user.

As shown, the movement data output by IMU 1502 may be received by processing unit 706. In this manner, processing unit 706 may use the movement data to determine (e.g., confirm) that intensity changes in the first and second signals (e.g., signals 902 or 1102) output by detectors 704 are in fact representative of a motion artifact, or that the intensity changes are representative of a motion artifact that should be compensated for.

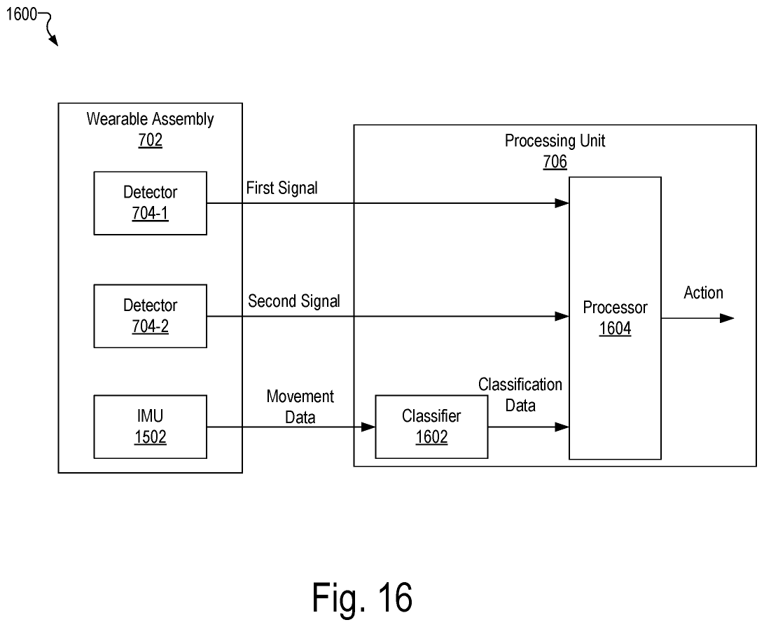

To illustrate, FIG. 16 shows an exemplary implementation 1600 of processing unit 706 in which processing unit 706 is configured to use movement data output by IMU 1302 to determine that intensity changes in the first and second signals output by detectors 704 are representative of a motion artifact. The various components shown in FIG. 16 as being included in processing unit 706 may be implemented by suitable combination of hardware, circuits, and/or software.

As shown, a classifier 1602 of processing unit 706 may receive the movement data and classify movement of the user based on the movement data. Classifier 1602 may output classification data representative of the classification. A processor 1604 included in processing unit 706 may receive the classification data, along with the first and second signals, and perform an action with respect to a motion artifact caused by the movement based on the classification of the movement.

Classifier 1602 may be implemented in any suitable manner. For example, classifier 1602 may be implemented by a machine learning model. The machine learning model may be supervised and/or unsupervised as may serve a particular implementation and may be configured to implement one or more decision tree learning algorithms, association rule learning algorithms, artificial neural network learning algorithms, deep learning algorithms, bitmap algorithms, and/or any other suitable data analysis technique as may serve a particular implementation. For example, the machine learning model may be implemented by one or more neural networks, such as one or more deep convolutional neural networks (CNN) using internal memories of its respective kernels (filters), recurrent neural networks (RNN), and/or long/short term memory neural networks (LSTM). In some examples, the machine learning model may be multi-layer. For example, the machine learning model may be implemented by a neural network that includes an input layer, one or more hidden layers, and an output layer.

Classifier 1602 may classify movement of a user in any suitable manner. For example, classifier 1602 may classify the movement as being acceptable, correctable, or uncorrectable. Any other suitable classification may be assigned to a movement as may serve a particular implementation.

An “acceptable” classification of a movement that causes a motion artifact may indicate that the movement is relatively minor such that that the motion artifact does not need to be compensated for. For example, movement caused by normal breathing may result in relatively small motion artifacts, and may therefore be classified as acceptable. In these cases, processor 1604 may abstain from compensating for the motion artifact.

A correctable classification of a movement that causes a motion artifact may indicate that the motion artifact may be successfully compensated for by subtracting a common mode signal from the first and second signals. For example, a sudden and relatively isolated movement of a head of the user may be a candidate for being classified as correctable. In these cases, processor 1604 may compensate for the motion artifact by subtracting a common mode signal from the first and second signals, as described herein.

An uncorrectable classification of a movement that causes a motion artifact may indicate that the motion artifact may not be successfully compensated for by subtracting a common mode signal from the first and second signals. For example, if the movement is performed in the context of various other movements, or if it may be relatively difficult to accurately identify a common mode signal for any reason, the movement may be classified as being uncorrectable. In these cases, processor 1604 may compensate for the motion artifact by discarding temporal portions of the first and second signals that include the motion artifact, as described herein.

Other classifications may be assigned to movements by processing unit 706 as may serve a particular implementation. For example, processing unit 706 may assign classifications to movements based on a scale of correctability or a confidence interval representing how accurate corrected data would be if one or more corrective measures were applied to the first and second signals.

In some examples, IMU 1502 may be configured to continuously stream movement data in a time synchronized manner with the first and second signals output by detectors 704. In these examples, processing unit 706 (e.g., processor 1604) may detect, based on the movement data, an occurrence of a movement event associated with the user that could possibly cause the motion artifact. The movement event may include, for example, a sudden movement of the user's head and/or any other movement that may cause a motion artifact.

Processing unit 706 may flag temporal portions of the first and second signals associated with the occurrence of the movement event for motion artifact analysis. This may be performed in any suitable manner. For example, processing unit 706 may record start and stop times for the temporal portions.

Processing unit 706 may then perform the motion artifact analysis with respect to the temporal portions of the first and second signals (e.g., in substantially real time as the first and second signals are output or at a later time). The motion artifact analysis may include any of the analysis operations described herein.

Processing unit 706 may then determine, based on the motion artifact analysis, that the first and second intensity changes are representative of the motion artifact. This may be performed in any of the ways described herein.

Additionally or alternatively, a pattern processor (e.g., that implements a machine learning model or other suitable processing heuristic) may be located on or communicatively coupled to IMU 1502 such that IMU 1502 does not stream any movement data under normal circumstances (e.g., during acceptable head motion). In these configurations, IMU 1502 may buffer historical movement data for a period of time (e.g., 10 seconds worth of movement data at any given time). If the pattern processor detects a movement event that is correctable, then IMU 1502 may transmit the contents of its buffer along with the first and second signals that the movement data can be used to compensate for the motion artifact in the first and second signals. If the pattern processor detects an uncorrectable error, processing unit 706 may discard the IMU data buffer. Alternatively, the IMU data buffer could be transmitted with the uncorrectable event to processing unit 706 to help with improving the correction algorithm (e.g., by training a machine learning model used by processing unit 706).

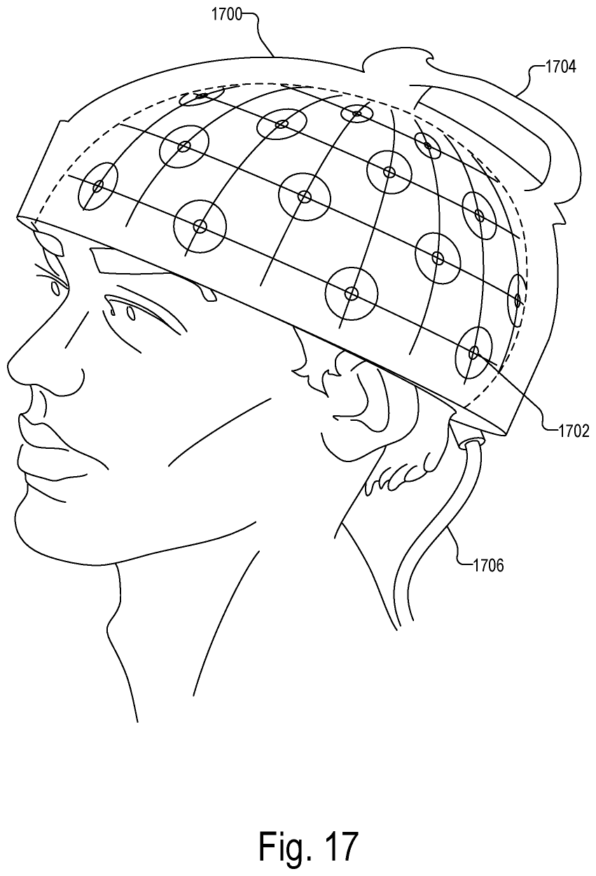

FIGS. 17-22 illustrate embodiments of a wearable device 1700 that includes elements of the optical detection systems described herein. In particular, the wearable devices 1700 shown in FIGS. 17-22 include a plurality of modules 1702, similar to the modules shown in FIG. 6 as described herein. For example, each module 1702 may include a source (e.g., source 604) and a plurality of detectors (e.g., detectors 606-1 through 606-6 and/or detectors 704-1 and 704-2). The wearable devices 1700 may each also include a controller (e.g., controller 112) and a processor (e.g., processor 108) and/or be communicatively connected to a controller and processor. In general, wearable device 1700 may be implemented by any suitable headgear and/or clothing article configured to be worn by a user. The headgear and/or clothing article may include batteries, cables, and/or other peripherals for the components of the optical measurement systems described herein.

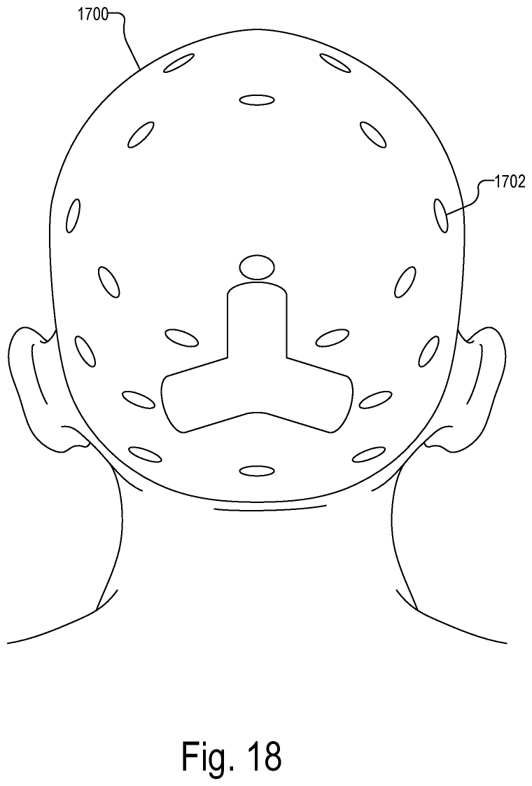

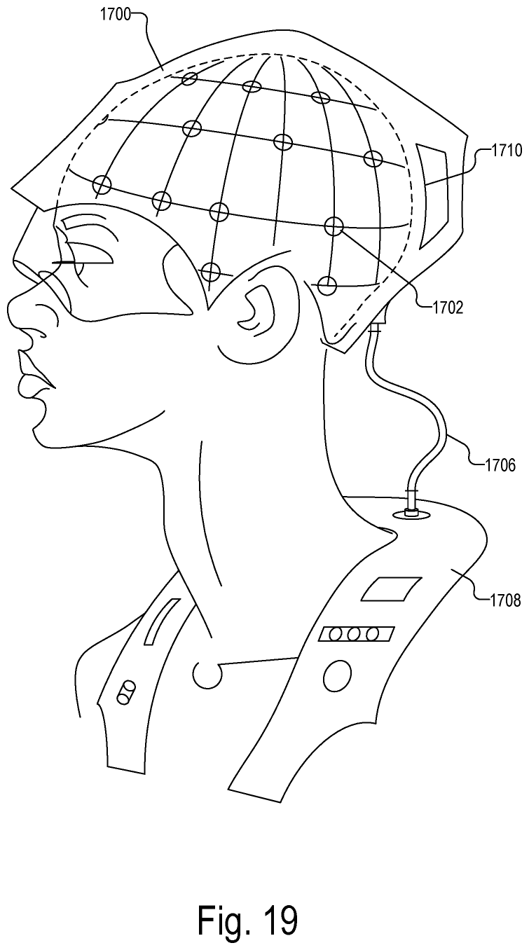

FIG. 17 illustrates an embodiment of a wearable device 1700 in the form of a helmet with a handle 1704. A cable 1706 extends from the wearable device 1700 for attachment to a battery or hub (with components such as a processor or the like). FIG. 18 illustrates another embodiment of a wearable device 1700 in the form of a helmet showing a back view. FIG. 19 illustrates a third embodiment of a wearable device 1700 in the form of a helmet with the cable 1706 leading to a wearable garment 1708 (such as a vest or partial vest) that can include a battery or a hub. Alternatively or additionally, the wearable device 1700 can include a crest 1710 or other protrusion for placement of the hub or battery.

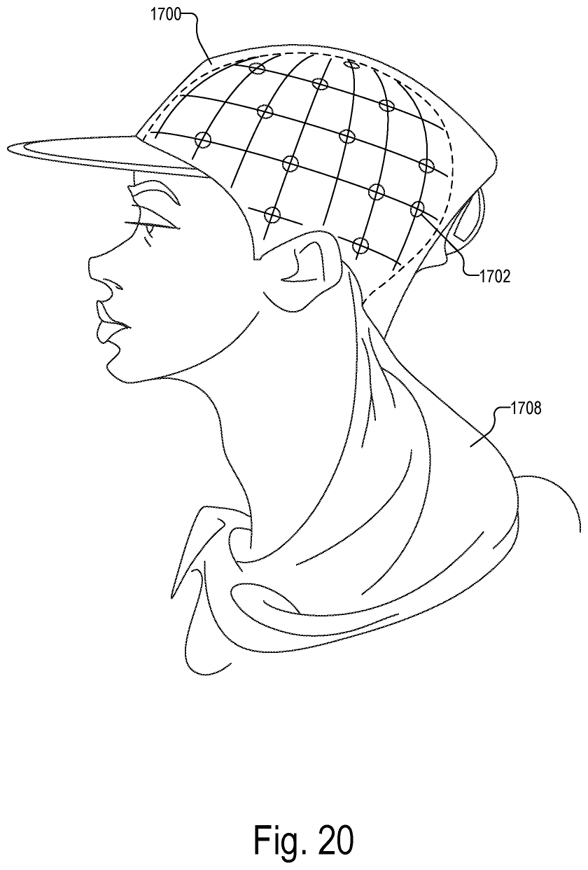

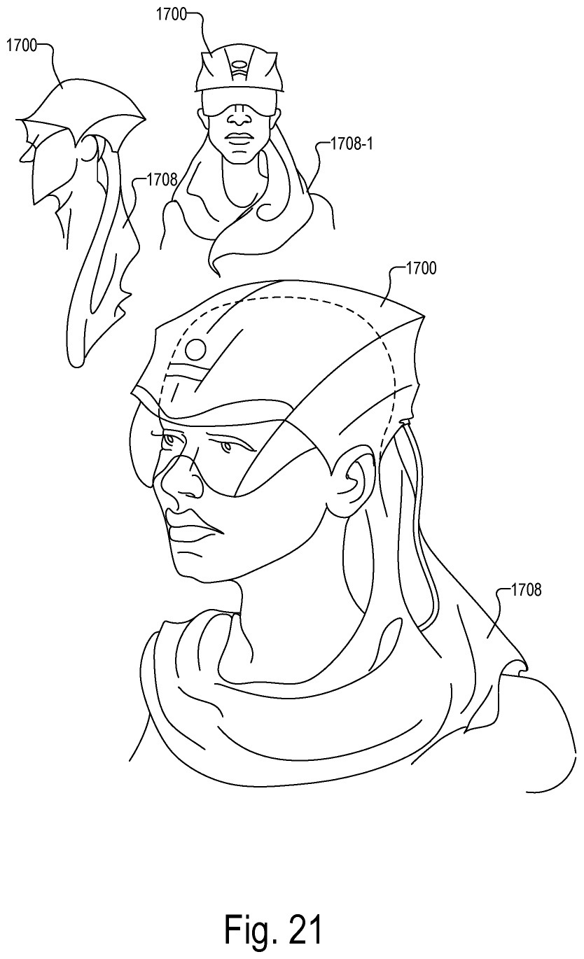

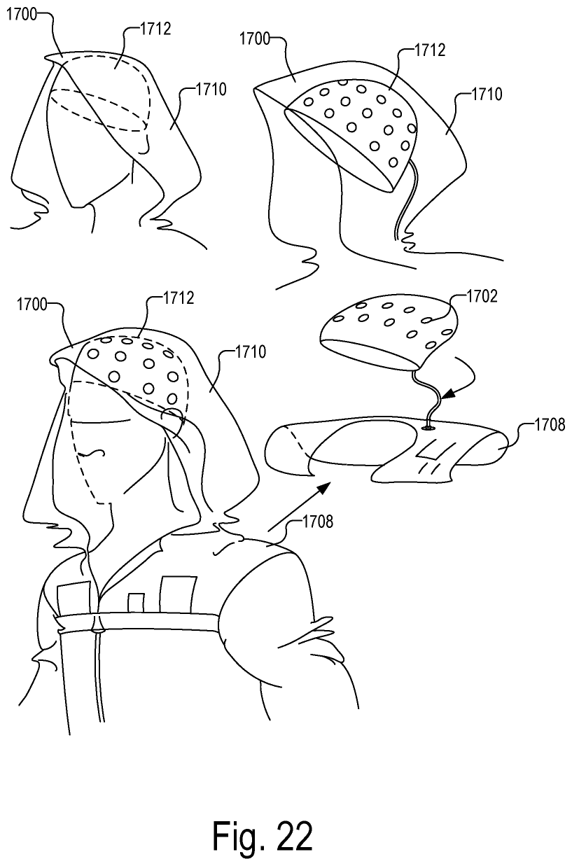

FIG. 20 illustrates another embodiment of a wearable device 1700 in the form of a cap with a wearable garment 1708 in the form of a scarf that may contain or conceal a cable, battery, and/or hub. FIG. 21 illustrates additional embodiments of a wearable device 1700 in the form of a helmet with a one-piece scarf 1708 or two-piece scarf 1708-1. FIG. 22 illustrates an embodiment of a wearable device 1700 that includes a hood 1710 and a beanie 1712 which contains the modules 1702, as well as a wearable garment 1708 that may contain a battery or hub.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

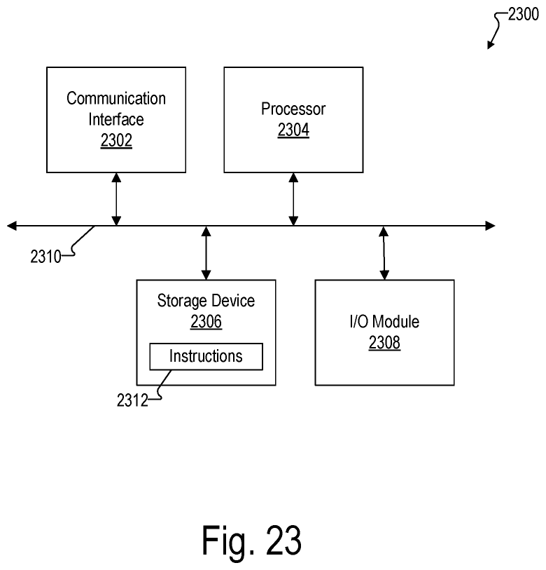

FIG. 23 illustrates an exemplary computing device 2300 that may be specifically configured to perform one or more of the processes described herein. Any of the systems, units, computing devices, and/or other components described herein may be implemented by computing device 2300.

As shown in FIG. 23, computing device 2300 may include a communication interface 2302, a processor 2304, a storage device 2306, and an input/output (“I/O”) module 2308 communicatively connected one to another via a communication infrastructure 2310. While an exemplary computing device 2300 is shown in FIG. 23, the components illustrated in FIG. 23 are not intended to be limiting. Additional or alternative components may be used in other embodiments. Components of computing device 2300 shown in FIG. 23 will now be described in additional detail.

Communication interface 2302 may be configured to communicate with one or more computing devices. Examples of communication interface 2302 include, without limitation, a wired network interface (such as a network interface card), a wireless network interface (such as a wireless network interface card), a modem, an audio/video connection, and any other suitable interface.

Processor 2304 generally represents any type or form of processing unit capable of processing data and/or interpreting, executing, and/or directing execution of one or more of the instructions, processes, and/or operations described herein. Processor 2304 may perform operations by executing computer-executable instructions 2312 (e.g., an application, software, code, and/or other executable data instance) stored in storage device 2306.

Storage device 2306 may include one or more data storage media, devices, or configurations and may employ any type, form, and combination of data storage media and/or device. For example, storage device 2306 may include, but is not limited to, any combination of the non-volatile media and/or volatile media described herein. Electronic data, including data described herein, may be temporarily and/or permanently stored in storage device 2306. For example, data representative of computer-executable instructions 2312 configured to direct processor 2304 to perform any of the operations described herein may be stored within storage device 2306. In some examples, data may be arranged in one or more databases residing within storage device 2306.

I/O module 2308 may include one or more I/O modules configured to receive user input and provide user output. I/O module 2308 may include any hardware, firmware, software, or combination thereof supportive of input and output capabilities. For example, I/O module 2308 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 2308 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 2308 is configured to provide graphical data to a display for presentation to a user. The graphical data may be representative of one or more graphical user interfaces and/or any other graphical content as may serve a particular implementation.

An illustrative system includes a wearable assembly configured to be worn by a user and comprising a first detector configured to detect a first set of photon arrival times and output a first signal representative of the first set of photon arrival times, and a second detector configured to detect a second set of photon arrival times and output a second signal representative of the second set of photon arrival times. The system further includes a processing unit configured to identify a first intensity change in the first signal, identify a second intensity change in the second signal, determine that the first and second intensity changes are time correlated, and determine, based on the determining that the first and second intensity changes are time correlated, that the first and second intensity changes are representative of a motion artifact caused by movement of the user.

An illustrative optical measurement system includes a plurality of modules each configured to be worn by a user and each comprising a plurality of detectors configured to detect photon arrival times and output signals representative of the photon arrival times, an inertial measurement unit configured to be worn by the user and detect movement by the user, and a processing unit configured to perform, based on the detected movement, an action with respect to the signals.

An illustrative system includes a memory storing instructions and a processor communicatively coupled to the memory and configured to execute the instructions to identify a first intensity change in a first signal output by a first detector included in a wearable assembly worn by a user, the first signal representative of a first set of photon arrival times detected by the first detector; identify a second intensity change in a second signal output by a second detector included in the wearable assembly, the second signal representative of a second set of photon arrival times detected by the second detector; determine that the first and second intensity changes are time correlated; and determine, based on the determining that the first and second intensity changes are time correlated, that the first and second intensity changes are representative of a motion artifact caused by movement of the user.

An illustrative method includes identifying, by a processing unit, a first intensity change in a first signal output by a first detector included in a wearable assembly worn by a user, the first signal representative of a first set of photon arrival times detected by the first detector; identifying, by the processing unit, a second intensity change in a second signal output by a second detector included in the wearable assembly, the second signal representative of a second set of photon arrival times detected by the second detector; determining, by the processing unit, that the first and second intensity changes are time correlated; and determining, by the processing unit based on the determining that the first and second intensity changes are time correlated, that the first and second intensity changes are representative of a motion artifact caused by movement of the user.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a wearable assembly configured to be worn by a user and comprising:
a first detector configured to detect a first set of photon arrival times and output a first signal representative of the first set of photon arrival times; and
a second detector configured to detect a second set of photon arrival times and output a second signal representative of the second set of photon arrival times; and
a processing unit configured to:
identify a first intensity change in the first signal;
identify a second intensity change in the second signal;
determine that the first and second intensity changes are time correlated; and
determine, based on the determining that the first and second intensity changes are time correlated, that the first and second intensity changes are representative of a motion artifact caused by movement of the user.

2. The system of claim 1, wherein the processing unit is further configured to compensate for the motion artifact.

3. The system of claim 2, wherein the compensating for the motion artifact comprises:
determining a common mode signal component included in both the first and second signals; and
subtracting the common mode signal component from the first and second signals.

4. The system of claim 2, wherein the compensating for the motion artifact comprises:
identifying a first temporal portion, within the first signal, that includes the motion artifact; and
discarding the first temporal portion from the first signal.

5. The system of claim 2, wherein the compensating for the motion artifact further comprises:
identifying a second temporal portion, within the second signal, that includes the motion artifact; and
discarding the second temporal portion from the second signal.

6. The system of claim 1, wherein:
the processing unit is further configured to determine that the first and second intensity changes are both greater than a threshold amount; and
the determining that the first and second intensity changes are time correlated is performed in response to the determining that the first and second intensity changes are both greater than the threshold amount.

7. The system of claim 1, wherein the determining that the first and second intensity changes are time correlated comprises determining that the first and second intensity changes are time correlated for at least a threshold amount of time.

8. The system of claim 1, further comprising:
an inertial measurement unit included in the wearable assembly and configured to output movement data associated with the wearable assembly;
wherein the determining that the first and second intensity changes are representative of the motion artifact is further based on the movement data.

9. The system of claim 8, wherein the inertial measurement unit comprises at least one of an accelerometer, a gyroscope, or a magnetometer.

10. The system of claim 8, wherein the processing unit is configured to:
classify, based on the movement data, the movement of the user that causes the motion artifact; and
perform, based on the classification of the movement, an action with respect to the motion artifact.

11. The system of claim 10, wherein:
the classifying of the movement comprises classifying the movement as being acceptable; and
the performing of the action comprises abstaining from compensating for the motion artifact.

12. The system of claim 10, wherein:
the classifying of the movement comprises classifying the movement as being correctable; and
the performing of the action comprises compensating for the motion artifact by subtracting a common mode signal from the first and second signals.

13. The system of claim 10, wherein:
the classifying of the movement comprises classifying the movement as being uncorrectable; and
the performing of the action comprises compensating for the motion artifact by discarding temporal portions of the first and second signals that include the motion artifact.

14. The system of claim 10, wherein the classifying is performed using a machine learning model.

15. The system of claim 8, wherein:
the inertial measurement unit is configured to continuously stream the movement data in a time synchronized manner with the first and second signals; and
the processing unit is further configured to:
detect, based on the movement data, an occurrence of a movement event associated with the user that could possibly cause the motion artifact,
flag temporal portions of the first and second signals associated with the occurrence of the movement event for motion artifact analysis, and
perform the motion artifact analysis with respect to the temporal portions of the first and second signals;
wherein the determining that the first and second intensity changes are representative of the motion artifact is based on the performing of the motion artifact analysis.

16. The system of claim 1, wherein the processing unit is included in the wearable assembly.

17. The system of claim 1, wherein the processing unit is not included in the wearable assembly.

18. The system of claim 1, wherein:
the wearable assembly comprises a particular module; and
the first and second detectors are both located on the particular module.

19. The system of claim 18, wherein the particular module comprises a light source configured to emit light that includes photons associated with the first and second sets of photon arrival times.

20. The system of claim 1, wherein:
the wearable assembly comprises a first module and a second module;
the first detector is located on the first module; and
the second detector is located on the second module.

21-43. (canceled)

44. A method comprising:
identifying, by a processing unit, a first intensity change in a first signal output by a first detector included in a wearable assembly worn by a user, the first signal representative of a first set of photon arrival times detected by the first detector;
identifying, by the processing unit, a second intensity change in a second signal output by a second detector included in the wearable assembly, the second signal representative of a second set of photon arrival times detected by the second detector;
determining, by the processing unit, that the first and second intensity changes are time correlated; and
determining, by the processing unit based on the determining that the first and second intensity changes are time correlated, that the first and second intensity changes are representative of a motion artifact caused by movement of the user.

45. The method of claim 45, further comprising compensating, by the processing unit, for the motion artifact.

46. The method of claim 44, wherein the compensating for the motion artifact comprises:
determining a common mode signal component included in both the first and second signals; and
subtracting the common mode signal component from the first and second signals.

47. The method of claim 45, wherein the compensating for the motion artifact comprises:
identifying a first temporal portion, within the first signal, that includes the motion artifact; and
discarding the first temporal portion from the first signal.

48. The method of claim 45, wherein the compensating for the motion artifact further comprises:
identifying a second temporal portion, within the second signal, that includes the motion artifact; and
discarding the second temporal portion from the second signal.

49. The method of claim 45, further comprising:
determining, by the processing unit, that the first and second intensity changes are both greater than a threshold amount; and
the determining that the first and second intensity changes are time correlated is performed in response to the determining that the first and second intensity changes are both greater than the threshold amount.

50. The method of claim 45, wherein the determining that the first and second intensity changes are time correlated comprises determining that the first and second intensity changes are time correlated for at least a threshold amount of time.

51. The method of claim 45, further comprising:
receiving, by the processing unit from an inertial measurement unit, movement data associated with the wearable assembly;
wherein the determining that the first and second intensity changes are representative of the motion artifact is further based on the movement data.

52. The method of claim 51, further comprising:
classifying, by the processing unit based on the movement data, the movement of the user that causes the motion artifact; and
performing, by the processing unit based on the classification of the movement, an action with respect to the motion artifact.

53. The method of claim 52, wherein:
the classifying of the movement comprises classifying the movement as being acceptable; and
the performing of the action comprises abstaining from compensating for the motion artifact.

54. The method of claim 52, wherein:
the classifying of the movement comprises classifying the movement as being correctable; and
the performing of the action comprises compensating for the motion artifact by subtracting a common mode signal from the first and second signals.

55. The method of claim 52, wherein:
the classifying of the movement comprises classifying the movement as being uncorrectable; and
the performing of the action comprises compensating for the motion artifact by discarding temporal portions of the first and second signals that include the motion artifact.

56. The method of claim 52, wherein the classifying is performed using a machine learning model.
